(19) United States
(12) Patent Application Publication
(10) Pub. No.: US 2015/0231289 A1
(43) Pub. Date: Aug. 20, 2015

(54) DISINFECTANT CAP FOR HYPODERMIC NEEDLES

(71) Applicant: 0736413 B.C. Ltd., Abbotsford (CA)

(72) Inventor: Garth T. Webb, Surrey (CA)

(21) Appl. No.: 14/422,409

(22) PCT Filed: Aug. 19, 2013
(86) PCT No.: PCT/CA2013/050638

§ 371 Date: Feb. 19, 2015

Related U.S. Application Data
(60) Provisional application No. 61/692,531, filed on Aug. 23, 2012.
(60) Provisional application No. 61/697,465, filed on Sep. 6, 2012.

(51) Int. Cl. A61L 2/18; A61M 5/32

(52) U.S. Cl. CPC A61L 2/18; A61M 5/3213; A61M 2209/10; A61L 2202/24

(57) ABSTRACT

A disinfectant cap for hypodermic needles includes a chamber that houses a liquid absorptive element that contains a biocide. The liquid absorptive element is fitted within the chamber to receive the dispensing tip of a hypodermic needle such that the needle tip penetrates the liquid absorptive material thereby imparting sustained antimicrobial action upon outer and inner regions of the hypodermic needle when the protective cap is secured in its closed configuration, and also to the interior of the syringe. The disinfectant cap is constructed such that the liquid absorptive element is retained within the chamber when the hypodermic needle is removed.

### CROSS REFERENCE TO RELATED APPLICATION

The present application claims the benefit, under 35 U.S.C. §119(e), of U.S. Provisional Patent Applications Ser. No. 61/692,531 filed Aug. 23, 2012 and 61/697,465 filed Sep. 6, 2012, both entitled “Disinfectant Cap for Hypodermic Needles” and both of which are incorporated herein by this reference.

### TECHNICAL FIELD

The present invention relates specifically to the disinfection of hypodermic needles and devices, such as ‘insulin pens’ that incorporate hypodermic needles.

### BACKGROUND

Various patent applications for disinfecting caps, or lids for liquid dispensers, arc of record. These disinfecting caps are designed to eliminate the need for chemical anti-microbials within a dispensed liquid. Generally, they house biocide-containing materials that are pressed against the dispensing port of a liquid dispensing tip that contains a one-way valve system, such as that described in Manesis published patent application no. US2006/0180613.

U.S. Pat. Nos. 4,392,859 and 4,507,118 to Dent disclose sterilising fitments relating to the sterilisation of hollow needles for livestock injections using an injecting gun. An enclosure fitted on the end of the gun contains a sponge impregnated with a sterilizing gel. The point of the needle passes through the enclosure before puncturing the skin of the animal and is withdrawn so the point of the needle passes back through the sponge. Theoretically, microorganisms are removed from the outer surface of the needles as it is withdrawn, but a problem with this process is that organisms may be harboured within interior spaces of the needle after withdrawal, thus compromising disease control.

There is a need therefore for a disinfectant cap whereby a hollow injection needle tip actually penetrates and lodges within a disinfecting medium after each use to impart chemical antimicrobial activity to vulnerable surfaces.

Safe disposal of bio-hazardous materials, such as contaminated hypodermic needles has been a public health issue for decades. Safe needle programs have been instituted by various health authorities, with mixed success. Both internal and external surfaces of used hypodermic needles must be disinfected for disposal to ensure public safety. Reduced costs, user-friendly operation and improved patient compliance are additional benefits that may be obtained by providing effective mechanical and chemical barriers to contamination. There is need for improvement.

The foregoing examples of the related art and limitations related thereto are intended to be illustrative and not exclusive. Other limitations of the related art will become apparent to those of skill in the art upon a reading of the specification and a study of the drawings.

### SUMMARY

The following embodiments and aspects thereof are described and illustrated in conjunction with systems, apparatus and methods which are meant to be exemplary and illustrative, not limiting in scope. In various embodiments, one or more of the above-described problems have been reduced or eliminated, while other embodiments are directed to other improvements.

A disinfectant cap for hypodermic needles is disclosed. The cap includes a chamber that houses a liquid absorptive element that contains a biocide. The liquid absorptive element is fitted within the chamber to receive the dispensing tip of a hypodermic needle, such that the needle tip penetrates the liquid absorptive material thereby imparting sustained antimicrobial action upon outer and inner regions of the hypodermic needle when the protective cap is secured in its closed configuration. The disinfectant cap is constructed such that the liquid absorptive element is retained within the chamber when the hypodermic needle is removed. According to one aspect a useful material for the absorptive element is an acrylic such as polyacrylamide.

In addition to the exemplary aspects and embodiments described above, further aspects and embodiments will become apparent by reference to the drawings and by study of the following detailed descriptions.

### BRIEF DESCRIPTION OF DRAWINGS

Exemplary embodiments are illustrated in referenced figures of the drawings. It is intended that the embodiments and figures disclosed herein are to be considered illustrative rather than restrictive.

In the drawings which disclose embodiments of the invention:

FIG. 1 is a vertical cross section of a first embodiment.

FIG. 2 is a vertical cross section of a second embodiment.

### DESCRIPTION

Throughout the following description, specific details are set forth in order to provide a more thorough understanding of the invention. However, the invention may be practised without these particulars. In other instances, well known elements have not been shown or described in detail to avoid unnecessarily obscuring the invention. Accordingly, the specification and drawings are to be regarded in an illustrative, rather than a restrictive, sense.

For purposes of this disclosure, “hypodermic needle” includes any hollow needle used to inject or extract substances into or from the body, including insulin pens.

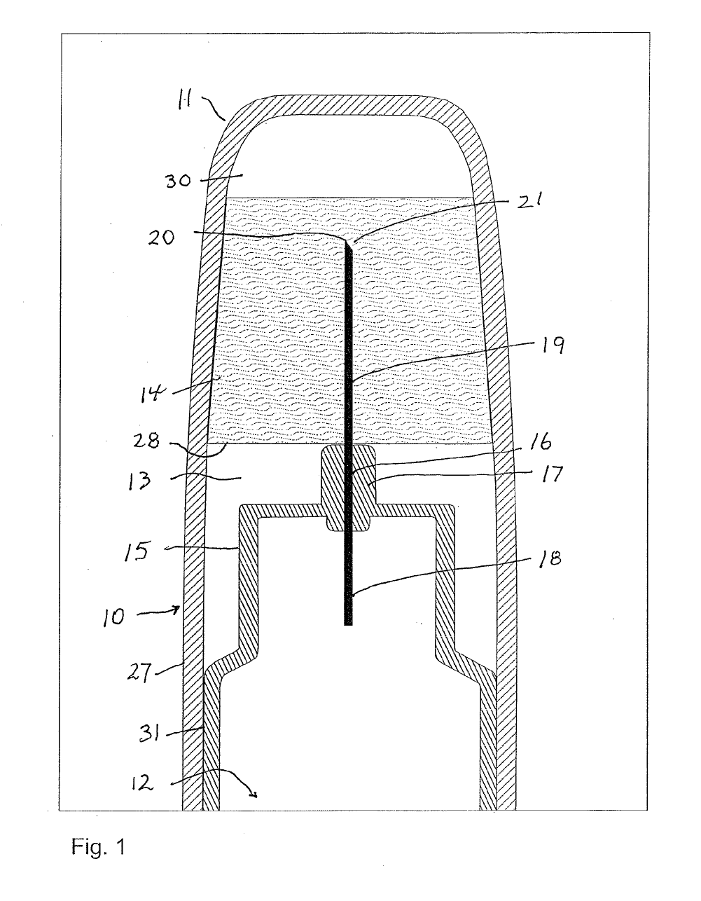

With reference to FIG. 1, a hollow cap 10, has a closed end 11 and an open end 12, and side walls 27 forming a hollow interior chamber 13. Chamber 13 retains a liquid absorptive clement 14 containing a chemical disinfectant or biocide. Side walls 27 seal circumferentially around the barrel 31 of a hollow syringe 15 when the cap 10 is in its closed configuration as shown, whether by friction fit or threaded connection. Hollow syringe 15 is attached to a hypodermic needle 16 by plastic hub 17 which may be a Luer™ taper connection. While the preferred embodiment has the cap 10 sealed around the barrel 31 of the syringe 15, it may also be sealingly secured to the hub 17.

The caudal shaft end 18 of hypodermic needle 16 extends into the hollow interior of syringe 15. In the closed configuration shown, the rostral shaft end 19 of hypodermic needle 16 penetrates the surface 28 of absorptive element 14, and extends further into the interior of absorptive element 14 as shown in FIG. 1. Absorptive element 14 may be retained within chamber 13 by a friction fit around its perimeter, adhesive, heat welding or other securement. Open confined space 30 within chamber 13 may be filled with liquid disinfectant. The walls 27 of disinfecting cap 10 may be constructed of optically transparent materials so that bright bio-compatible visual indicators such as vitamin B12, riboflavin and carotenes may be mixed within the disinfecting solution to provide visual confirmation of the presence of the disinfecting solution.

The sharp tip 20 of the hypodermic needle 16 and the rostral shaft end 19 are lodged within the interior of the absorptive element 14 when cap 10 is secured in its closed configuration, as shown. The interior bore 21 of the hypodermic needle 16 connects the interior of hollow syringe 15 with the interior of absorptive element 14.

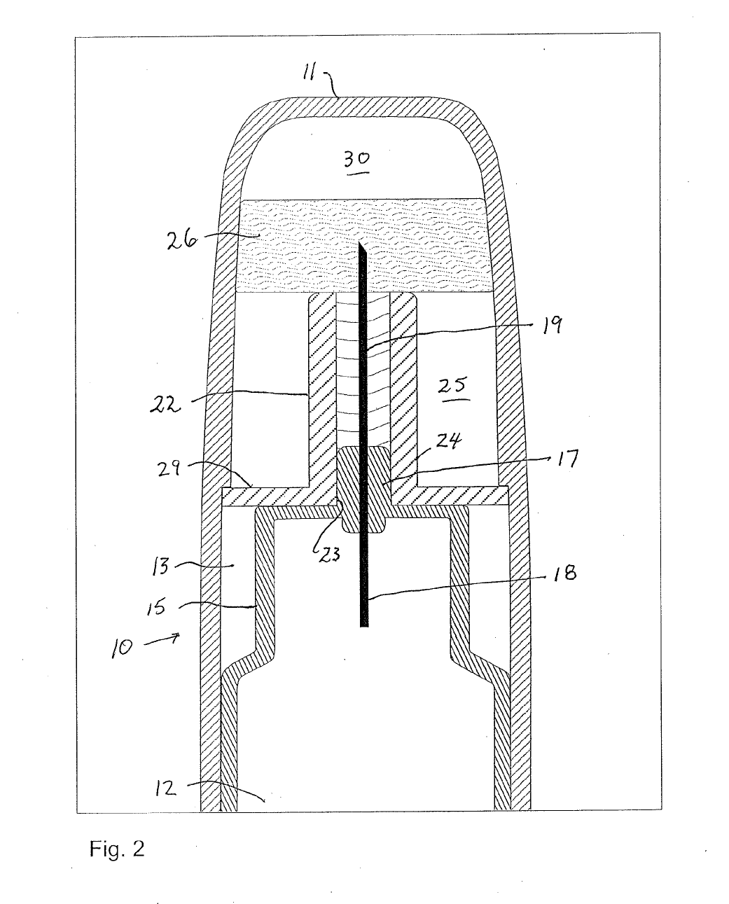

FIG. 2 shows similar detail except that a rigid retainer 22 is added to define interior compartments within chamber 13. Cylindrical shaft 24 extending from annular disc 29 receives the hub 17 through aperture 23. Cylindrical shaft 24 may be cut away on a longitudinal plane to create a hemi-cylindrical shape to visually expose hypodermic needle 16. Ancillary space 25 is defined by the space between absorptive element 14 and retainer 22. Optional reservoir 30 is defined by the space between closed end 11 and absorptive element 14. Ancillary space 25 and reservoir 30 may store liquid disinfectants, such as ethyl alcohol, to disinfect exposed portions of rostral shaft 19 and hub 17. These stored liquid disinfectants also replace chemical agents within absorptive element 14 that are lost due to migration of molecules into interior bore 21. A seal by friction fit between hub 17 and aperture 23 is engaged when cap 10 is secured in its closed configuration, as shown.

Aperture 23 within rigid retainer 22 limits the potential loss of liquid out of shaft 24 by surface tension, such as at the intersection with the surface of absorptive element 26, when cap 10 is disengaged from syringe 15. The sharp tip 20 and the segment of rostral shaft 19 that is lodged within the absorptive element 14, 26 are subject to the molecular dynamics of disinfectants therein. Given time, the internal bore 21 of hypodermic needle 16 and the internal space of hollow syringe 15 are subject to these same molecular dynamics. Liquid within ancillary space 25 and reservoir 30 may transfer fortified concentrations of chemical agents into absorptive element 14, 26 to replace molecules lost to migration within interior bore 21. Optionally, rostral shaft 19 may extend through absorptive element 14, 26 into optional reservoir 30. Surfaces of rostral shaft 19 are thus disinfected by absorptive element 14, 26 whereas sharp tip 20 and interior bore 21 are exposed to the contents of optional reservoir 30.

Surface tension is the natural phenomenon whereby cohesive forces between liquid molecules resist external force. These cohesive forces may be altered or dominated and ultimately replaced by the process of crystallization. Liquids may thus be converted into gels, soft solids and rigid matter. Very often, the process is reversible. Most varieties of gels and soft solids are liquid absorptive.

Some gels are made of materials, such as silicon, that are non-absorptive to aqueous liquids. Gels made from these materials may be infused with disinfecting materials that are water soluble, making the final composite mildly water absorptive. Once dissolved, the disinfecting molecules are free to migrate. Some gels are made of water soluble materials, such as hyluronate. Gels such as these, infused with disinfectants, release disinfecting molecules either through diffusion through their material matrixes or through direct release as their matrixes are dissolved by an external source of aqueous liquid, such as liquid within the bore of a used hypodeunic needle.

Within the continuum of material matrixes available, the ability of any one compound to maintain a fixed position within the disinfecting cap described herein, is dependent upon a number of factors, an important factor being the inverse of surface area. The surface area of the open end of the disinfecting cap is relatively small for most applications, thus, the selection for molecularly absorptive materials for construction of liquid absorptive elements is relatively unrestricted.

Some liquid absorptive materials consist of structural mesh-works that allow liquids to flow through. Fine structural elements within absorptive element 14, 26 regulate the movement of antimicrobial agents. Spaces between these structural elements are normally referred to as ‘pores’. Materials such as sponges and cellulose fiber have large pore sizes, unless they are tightly compressed. Tightly compressed fibers, such as these, do not make good absorptive elements for disinfection of hypodermic needles as they are prone to damage them. In a non-compressed state, they do not cause damage to hypodermic needles. Generally non-compressed sponges and cellulose fiber do not impede the diffusion of disinfectants upon target surfaces and are especially desirable for single-use hypodermic needles.

Unused hypodermic needles are normally filled with air. Air within the internal bore of hypodermic needles produces an ‘air-lock’ that prevents the migration of liquid disinfectant molecules therein. Air-locks thus prevent chemical contamination of unused hypodermic needles from chemicals within their disinfecting caps. Once a hypodermic needle is used, liquid remaining from the injection of liquid transferred from the interior of the attached syringe fills the internal bore of the hypodermic needle, leaving it susceptible to biological contamination. Outer surfaces of the hypodermic needle are invariably coated with organic matter and microorganisms, creating an obvious bio-hazard.

Disinfecting caps 10 according to the invention with absorptive elements 14, 26 having large pore sizes are best suited for use with single-use hypodermic needles. When these types of disinfecting caps 10 are employed, disinfecting molecules are quickly mobilized to protect both the external surfaces and internal spaces within hypodermic needles 16. Eventually, molecular migration of disinfectants spreads into the hollow interior of the attached syringe 15, making it safe for disposal.

Absorptive polymeric materials that are soft in texture do not cause damage to hypodermic needles. These materials are particularly useful for multi-use applications, such as ‘insulin pens’. The pore size of absorptive polymeric materials may change greatly depending upon their nature and upon their environments. One particularly useful material is an acrylic such as polyacrylamide. In its dried state, polyacrylamide is a relatively firm compound with a tight matrix and a small pore size. When hydrated, its texture changes. It softens to allow even blunt needles to penetrate. Its pores enlarge from small semi-permeable size to large sizes that are large enough to allow free diffusion of very large molecules. The process is easily reversed. Its swollen matrix shrinks back to normal size with small ports through evaporation or with the addition of alcohol or electrolytes, such as sodium chloride.

Various disinfectants, either with or without osmotic agents, may be matched with polymeric absorptive elements, such as polyacrylamide, to safely regulate the flow and migration of disinfectant molecules within the interior bore of a hypodermic needle 16 without causing significant chemical contamination of the dispensed product, for a predetermined period of time.

Hypodermic needle tips 20 that penetrate and lodge within biocide-containing liquid absorptive polymeric elements 14, 26 experience restricted exposure to disinfectants. Internal surfaces and the liquid content within the internal bore 21 of hypodermic needles 16 are subject to a myriad of molecular dynamics that are affected by alcohol concentration, pH, osmotic forces, electrolyte concentration, pore size, material interactions and fluid dynamics. The migration of disinfectant molecules within the internal bore 21 may be tailored to suit specific requirements by controlling one or more of these factors. Disinfectant molecules within the needle bore 21 diffuse toward the caudal end 18 of the needle whereas osmotic agents may be employed to draw water molecules in either direction. This action may be used to modify the migration of disinfectants within the needle bore 21 as desired.

Disinfectants for the disinfecting cap 10 include but are not limited to biguanides, quaternary ammonium polymers, iodine compounds and alcohols. Osmotic agents include but are not limited to salts, such as sodium chloride and potassium chloride as well as small molecular proteins. Materials used to construct the liquid absorptive element 14, 26 include hydrophilic acrylics, sponges, cellulose fiber, gels and viscosity agents such as hyluronate.

The present invention does not attempt to eliminate the presence of chemical anti-microbials within a dispensed liquid product; rather, it focuses upon the control and migration of chemical disinfectants upon vulnerable surfaces of liquid dispensing devices, such as hypodermic needles, to mitigate risk and improve patient compliance. The present invention thus includes varieties of liquid absorptive materials and chemical disinfectants, to control molecular dynamics and the migration of chemical substances within and upon contaminated hypodermic needles to destroy microorganisms.

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

What is claimed is:

1. A disinfectant cap for hypodermic needles comprising:
i) a hollow body having a closed end, an open end and a wall enclosing a chamber; and
ii) a liquid absorptive element within said chamber, said liquid absorptive element containing a biocide;
wherein said liquid absorptive element is adapted to receive the penetrating tip of a hypodermic needle, such that the needle tip penetrates the liquid absorptive material thereby imparting sustained disinfecting action upon outer and inner regions of the hypodermic needle when said hypodermic needle is inserted into said open end and releasably secured within said chamber.

2. The disinfectant cap of claim 1 wherein said cap is constructed such that the liquid absorptive element is retained within said chamber when said hypodermic needle is removed.

3. The disinfectant cap of claim 1 wherein said absorptive element comprises a material selected from the group consisting of hydrophilic acrylics, sponges, cellulose fiber, gels and viscosity agents such as hyluronate.

4. The disinfectant cap of claim 3 wherein said absorptive element comprises a hydrophilic acrylic.

5. The disinfectant cap of claim 4 wherein said absorptive element comprises polyacrylamide.

6. The disinfectant cap of claim 3 wherein said absorptive element comprises a non-water absorptive gel infused with a water soluble disinfectant, whereby said absorptive element is rendered water absorptive and releases said disinfectant once said disinfectant is dissolved.

7. The disinfectant cap of claim 6 wherein said non-absorptive gel comprises silicon.

8. The disinfectant cap of claim 3 wherein said absorptive element comprises a gel formed from water soluble materials, such as hyluronate, and wherein said gel is infused with disinfectant to thereby release said disinfectant when said gel is dissolved by an external source of water.

9. The disinfectant cap of claim 3 wherein said sponges and/or cellulose fiber have a large pore size.

10. The disinfectant cap of claim 4 wherein said hydrophilic acrylic is hydrated to provide a large pore size.

11. The disinfectant cap of claim 10 wherein said pore size of said hydrophilic acrylic is varied by addition of alcohol or electrolytes.

12. The disinfectant cap of claim 1 or 4 wherein said absorptive element further comprises, alcohol, osmotic agent or electrolyte to vary the degree of migration of said biocide within said hypodermic needle.

13. The disinfectant cap of claim 1 or 4 further comprising an osmotic agent to draw water molecules in either direction in said hypodermic needle.

14. The disinfectant cap of claim 12 or 13 wherein said osmotic agent is selected from the group consisting of salts, such as sodium chloride and potassium chloride, and small molecular proteins.

15. The disinfectant cap of claim 1 wherein said wall of said disinfecting cap comprises optically transparent material.

16. The disinfectant cap of claim 15 wherein said biocide comprises visual indicators to provide visual confirmation of the presence of a disinfecting solution.

17. The disinfectant cap of claim 16 wherein said visual indicators are bright bio-compatible visual indicators selected from the group consisting of vitamin B12,riboflavin and carotenes.

18. The disinfectant cap of claim 1 wherein said biocide is selected from the group consisting of biguanides, quaternary ammonium polymers, iodine compounds and alcohols.

19. The disinfectant cap of claim 1 wherein said biocide is a disinfectant.

20. The disinfectant cap of claim 1 further comprising a first liquid reservoir formed by a space between said closed end and said absorptive element.

21. The disinfectant cap of claim 20 wherein said first liquid reservoir receives a liquid disinfectant.

22. The disinfectant cap of claim 1 further comprising a needle-retaining element comprising an annular disc secured in said chamber having a central aperture for receiving the end of a hypodermic needle mounted on a syringe.

23. The disinfectant cap of claim 22 wherein said central aperture sealingly receives a hub of a syringe having a hypodermic needle mounted therein.

24. The disinfectant cap of claim 22 wherein said needle-retaining element further comprises a cylindrical element secured to said annular disc and having a central hollow shaft communicating with said central aperture for receiving said needle.

25. The disinfectant cap of claim 24 further comprising a second liquid reservoir defined by a space between said absorptive element and said annular disc.

26. In combination, a hypodermic needle and the disinfectant cap of any one of claims 1-25.

27. In combination, a hypodermic needle, a syringe and the disinfectant cap of any one of claims 1-25, said hypodermic needle being mounted on said syringe, wherein said cap is sized to be releasably secured to the barrel of said syringe.
